(19) United States
(12) Patent Application Publication
(10) Pub. No.: US 2023/0303142 A1
(43) Pub. Date: Sep. 28, 2023

(54) OPERATION DEVICE

(71) Applicant: Honda Motor Co., Ltd., Tokyo (JP)

(72) Inventors: Jun Inada, Saitama (JP); Ritsuya Oshima, Saitama (JP); Hiroshi Iwakami, Saitama (JP); Hiroshi Gomi, Saitama (JP); Shinichiro Kobashi, Saitama (JP); Makoto Hasegawa, Saitama (JP)

(73) Assignee: Honda Motor Co., Ltd., Tokyo (JP)

(21) Appl. No.: 18/184,678

(22) Filed: Mar. 16, 2023

(30) Foreign Application Priority Data
Mar. 25, 2022 (JP) 2022-049848

(51) Int. Cl. B62B 3/12; B62B 5/00

(52) U.S. Cl. CPC B62B 3/12; B62B 5/0033

(57) ABSTRACT

An operation device 5 includes: a base 40; an operation part 45, extending in a left-right direction; and a pair of left and right first connection parts 50, provided between the base and the operation part, and disposed apart from each other in the left-right direction. Each first connection part includes: a first end 56, connected to the base; a second end 55, connected to the operation part; a first elastic member 63, connecting the second end with respect to the first end to be displaceable in a front-rear direction; and a first distance sensor 65A, 65B, measuring a relative distance of the second end with respect to the first end in the front-rear direction.

### CROSS-REFERENCE TO RELATED APPLICATION

This application claims the priority benefit of Japan application serial No. 2022-049848, filed on Mar. 25, 2022. The entirety of the above-mentioned patent application is hereby incorporated by reference herein and made a part of this specification.

### BACKGROUND

### Technical Field

The disclosure relates to an operation device.

### Description of Related Art

Patent Document 1 discloses a truck supporting an X-ray device. The truck has a handle held by the user and wheels. The control device of the truck assist-drives the wheels based on a load input to the handle.

### Prior Art Documents

### [Patent Document]

[Patent Document 1] Japan Patent No. 5820376

Force sensors that detect multi-directional loads applied to a handle are generally expensive. Therefore, it is desired to lower the cost of an operation device.

### SUMMARY

An aspect of the disclosure provides an operation device, including: a base; an operation part, extending in a left-right direction; and a pair of left and right first connection parts, provided between the base and the operation part, and disposed apart from each other in the left-right direction. Each of the first connection parts includes: a first end, connected to the base; a second end, connected to the operation part; a first elastic member, connecting the second end with respect to the first end to be displaceable in a front-rear direction; and a first distance sensor, measuring a relative distance of the second end with respect to the first end in the front-rear direction.

### BRIEF DESCRIPTION OF THE DRAWINGS

FIG. 1 is a perspective view illustrating a truck according to an embodiment.

FIG. 2 is a plan view of a truck.

FIG. 3 is a cross-sectional view of an omnidirectional wheel.

FIG. 4 is a side view of a main wheel.

FIG. 5 is a perspective view of an operation device.

FIG. 6 is a vertical cross-sectional view illustrating a stopper.

FIG. 7 is a horizontal cross-sectional view illustrating a stopper.

FIG. 8 is a side view illustrating a first distance sensor when an operation force is not applied.

FIG. 9 is a side view illustrating the first distance sensor when a forward operation force is applied.

FIG. 10 is a front view illustrating a second sensor when an operation force is not applied.

FIG. 11 is a front view illustrating the second distance sensor when a rightward operation force is applied.

FIG. 12 is a block diagram illustrating a control device of a truck.

### DESCRIPTION OF THE EMBODIMENTS

The disclosure provides an inexpensive operation device.

An aspect of the disclosure for solving the above issue provides an operation device (5). The operation device includes: a base (40); an operation part (45), extending in a left-right direction; and a pair of left and right first connection parts (50), provided between the base and the operation part, and disposed apart from each other in the left-right direction. Each of the first connection parts includes: a first end (56), connected to the base; a second end (55), connected to the operation part; a first elastic member (63), connecting the second end with respect to the first end to be displaceable in a front-rear direction; and a first distance sensor (65A, 65B), measuring a relative distance of the second end with respect to the first end in the front-rear direction.

According to the aspect, an operation toward the front-rear direction and a turning operation performed by the user can be detected by the left and right side first distance sensors. Since the distance sensors can be acquired at a lower cost, an inexpensive operation device can be provided.

In the above aspect, at least one second elastic member (64) connecting each of the first ends and the base to be displaceable in the left-right direction and a second distance sensor (75) measuring a relative distance of one of the first ends with respect to the base in the left-right direction may be further provided.

According to the aspect, the operation force of the user in the left-right direction can be detected.

In the above aspect, it may also be that the second end of the first connection part disposed above the second distance sensor is fixed to the operation part to be not displaceable in the left-right direction.

According to the aspect, the displacement of the operation part in the left-right direction can be efficiently transmitted to the second elastic member.

In the above aspect, it may also be that the second end of the first connection part different from the first connection part disposed above the second distance sensor is connected to the operation part to be displaceable in the left-right direction.

According to the aspect, the measurement accuracy of the second distance sensor can be facilitated.

In the above aspect, it may also be that each of the first distance sensors is provided with: a first piece (67), conductive and extending from the second end toward the corresponding first end; and a first eddy current sensor (69), provided at the first end and corresponding to the first piece in the front-rear direction.

According to the aspect, the first distance sensor can be formed in a cost-effective manner. Moreover, the size and the measurement distance of the first distance sensor can be set properly.

In the above aspect, it may also be that each of the second distance sensors is provided with: a second piece (77), conductive and extending from the first end toward the base; and a second eddy current sensor (79), provided at the base and corresponding to the second piece in the left-right direction.

According to the aspect, the second distance sensor can be formed in a cost-effective manner. Moreover, the environment resistance and the service time of the second distance sensor can be facilitated.

In the above aspect, a stopper (48) may be further provided. The stopper is provided at the base and defining a movement range of the operation part through abutting against the operation part.

According to the aspect, the movement range of the operation part can be defined, and the operation force can be suppressed from being applied to the first distance sensor.

In the above aspect, it may also be that the stopper is provided between the second ends at left and right, and the stopper is provided with a through hole (53) extending in the left-right direction and accommodating the operation part.

According to the aspect, the left and right first contact parts and the stopper can be disposed in a compact manner.

In the above aspect, at least one second elastic member connecting each of the second ends and the operation part to be displaceable in the left-right direction and a second distance sensor, measuring a relative distance of the operation part with respect to one of the second ends in the left-right direction may be further provided.

According to the aspect, the operation force of the user in the left-right direction can be detected.

According to the above configuration, an inexpensive operation device can be provided.

Embodiments of a truck suitable for an operation device according to the disclosure will be described below with reference to the drawings. Hereinafter, each direction is defined with the truck as a reference.

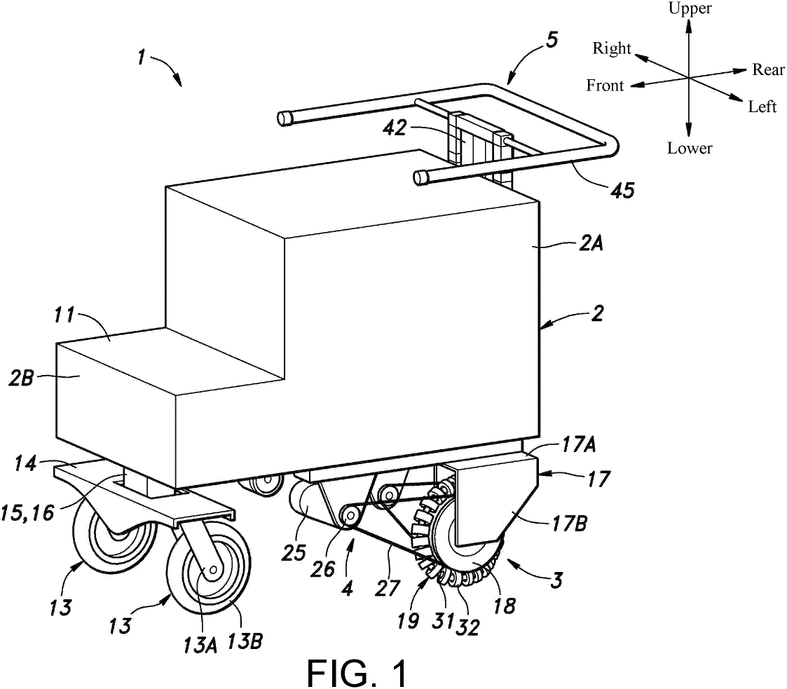

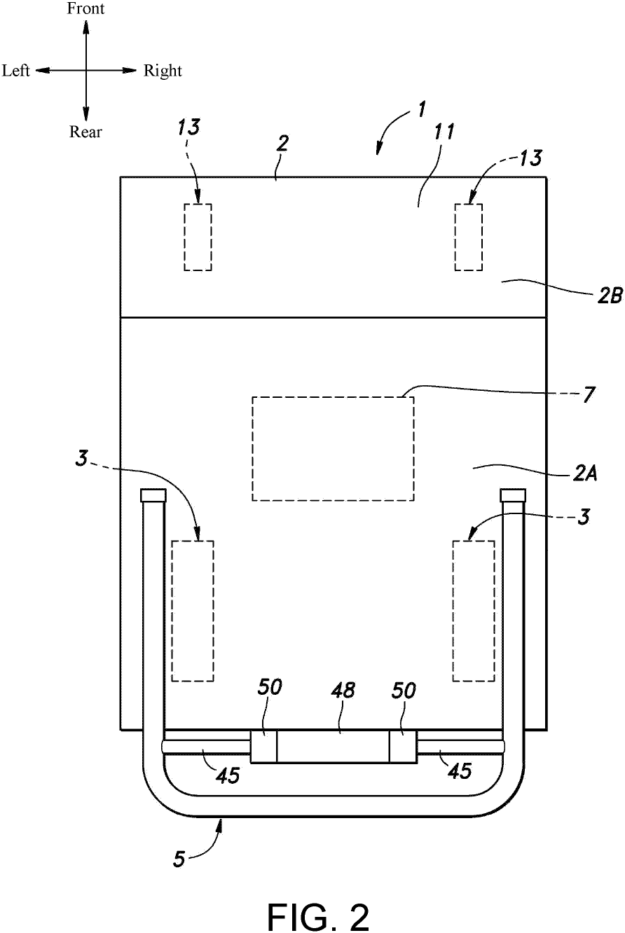

As shown in FIGS. 1 and 2, a truck 1 includes: a body 2; at least one omnidirectional wheel 3, provided on the body 2 and moving the body 2 in all directions along a floor surface; a drive unit 4, driving each omnidirectional wheel 3; an operation device 5; and a control device 7, controlling the drive unit 4.

The body 2 extends in a front-rear direction. A rear part 2A of the body 2 extends above a front part 2B. The front part 2B of the body 2 is provided with a support stand 11 for supporting another device. Examples of the device supported by the support stand 11 include an inspection device such as an X-ray scanner. The device may be fastened to the support stand 11. The control device 7, a battery, and various sensors may be provided inside the rear part 2A of the body 2.

In the present embodiment, a pair of omnidirectional wheels 3 are provided at a lower portion of the rear part 2A of the body 2. Left and right casters 13 are supported by a lower portion of the front part 2B of the body 2 via a suspension. The suspension includes: an arm 14, arranged below the body 2 and extending in a left-right direction; and a spring 15 and a shock absorber 16, arranged between the body 2 and the arm 14. Each caster 13 is arranged below left and right ends of the arm 14. Each caster 13 includes: a fork 13A, rotatably coupled to the arm 14 about an axis extending in an up-down direction; and a wheel 13B, rotatably supported by the fork 13A about an axis extending in a horizontal direction. The fork 13A rotates freely with respect to the arm 14, and the wheel 13B rotates freely with respect to the fork 13A.

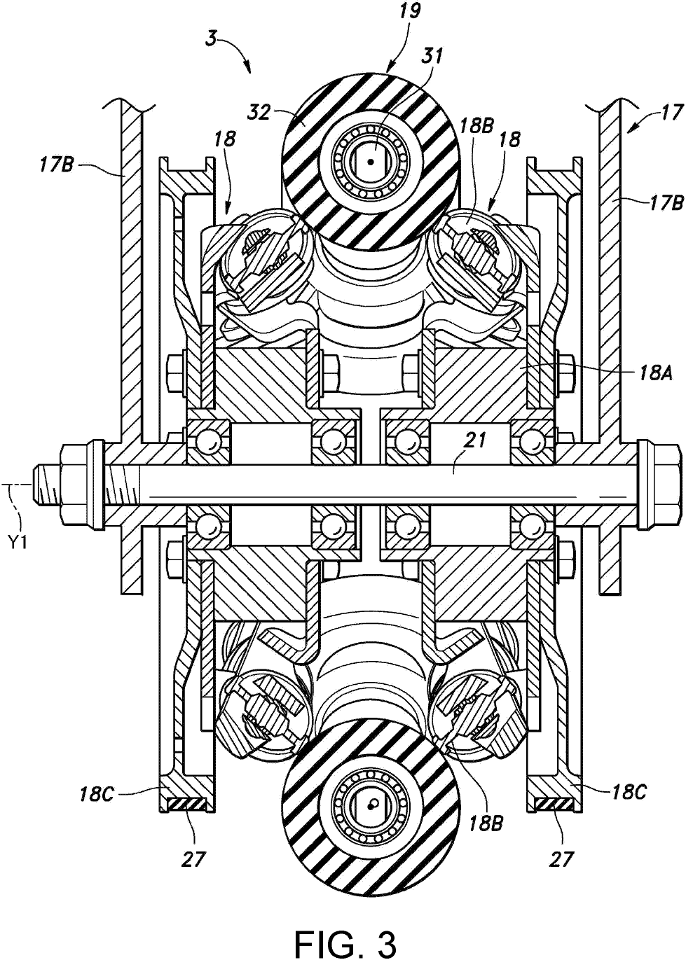

As shown in FIG. 2, the pair of omnidirectional wheels 3 are arranged with an interval therebetween in the left-right direction. In the present embodiment, the pair of omnidirectional wheels 3 are arranged at the lower left and lower right of the rear part 2A of the body 2. As shown in FIG. 3, each omnidirectional wheel 3 includes: a frame 17; a pair of drive discs 18, rotatably supported by the frame 17; and a main wheel 19 of an annular shape, arranged between the pair of drive discs 18.

As shown in FIG. 1 and FIG. 3, the frame 17 has: a frame upper part 17A, coupled to a lower part of the body 2; and a pair of frame sides 17B, extending downward from both left and right ends of the frame upper part 17A. A support shaft 21 extending in the left-right direction is extended across lower ends of the pair of frame sides 17B. The pair of drive discs 18 are rotatably supported by the support shaft 21. The pair of drive discs 18 rotate about an axis Y1 of the support shaft 21. A position of each drive disc 18 in the left-right direction with respect to the support shaft 21 is restricted. The drive discs 18 face each other with a distance therebetween in the left-right direction.

The drive disc 18 is arranged on each of both sides of the main wheel 19 of an annular shape, and applies a frictional force to the main wheel 19 to rotate the main wheel 19 about a central axis and about an annular axis. The drive disc 18 includes: a base 18A of a disc shape, rotatably supported by the frame 17; and a plurality of drive rollers 18B, rotatably supported to be inclined with respect to each other at an outer periphery of the base 18A and being in contact with the main wheel 19. The base 18A is arranged coaxially with the support shaft 21.

A driven pulley 18C is provided on each of opposite surfaces of each drive disc 18. The driven pulley 18C is provided coaxially with the drive disc 18. The drive unit 4 is provided at a lower part of the body 2 and includes a plurality of electric motors 25 corresponding to each drive disc 18. In the present embodiment, four electric motors 25 are provided corresponding to four drive discs 18. A drive pulley 26 is provided on an output shaft of each electric motor 25. The drive pulley 26 and the driven pulley 18C corresponding to each other are connected by a belt 27. By rotating each electric motor 25 independently of each other, each drive disc 18 rotates independently of each other.

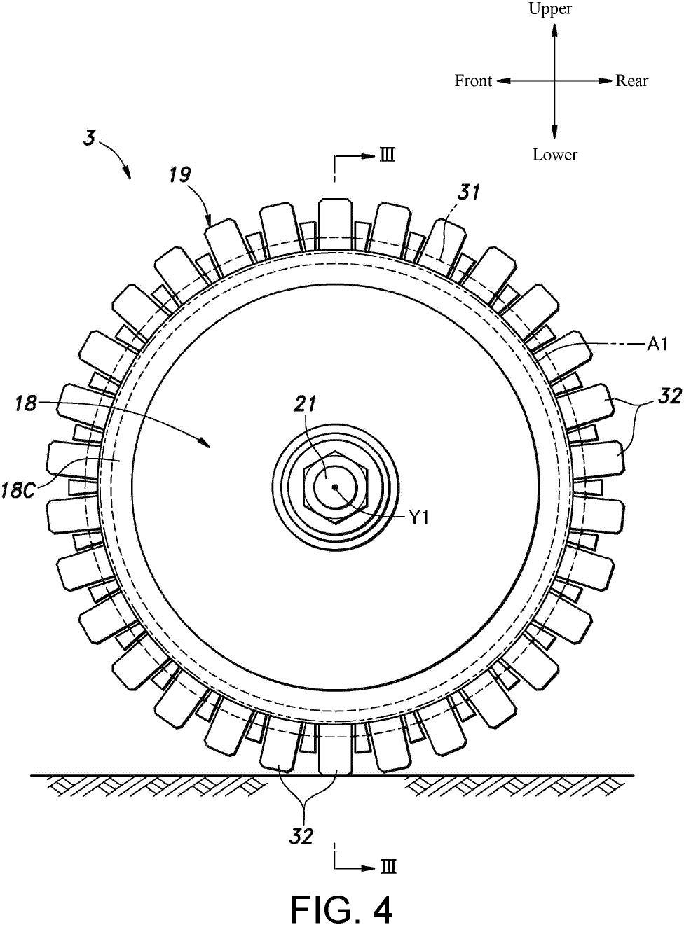

As shown in FIG. 4, the main wheel 19 is of an annular shape, is arranged between and coaxially with the pair of drive discs 18, is in contact with the plurality of drive rollers 18B, and is rotatable about the central axis and about the annular axis. The main wheel 19 includes a core body 31 of an annular shape and a plurality of driven rollers 32 rotatably supported by the core body 31. The plurality of driven rollers 32 are arranged at equal intervals in a circumferential direction of the core body 31. Each driven roller 32 is supported by the core body 31 so as to be rotatable about an axis A1 (annular axis) of the core body 31 of an annular shape. Each driven roller 32 is able to rotate at each position with respect to the core body 31 about a tangent of the core body 31. Each driven roller 32 rotates with respect to the core body 31 upon receiving an external force.

The main wheel 19 is arranged along an outer periphery of the pair of drive discs 18 and is in contact with the plurality of drive rollers 18B provided on each drive disc 18. The drive roller 18B of each drive disc 18 is in contact with an inner periphery of the main wheel 19 and clamps the main wheel 19 from both left and right sides. By contact with the inner periphery of the main wheel 19, displacement of the drive roller 18B of the left and right drive discs 18 in a radial direction about the axis Y1 of the drive disc 18 is restricted. Accordingly, the main wheel 19 is supported by the left and right drive discs 18, and the central axis of the main wheel 19 (core body 31) is arranged coaxially with the axis Y1 of the left and right drive discs 18. The main wheel 19 is in contact with the plurality of drive rollers 18B of the left and right drive discs 18 at the plurality of driven rollers 32.

In each omnidirectional wheel 3, if the pair of drive discs 18 rotate in the same direction at the same rotation speed, the main wheel 19 rotates together with the pair of drive discs 18. That is, the main wheel 19 rotates forward or rearward about its own rotation axis that coincides with the axis Y1. At this time, the drive roller 18B of the drive disc 18 and the driven roller 32 of the main wheel 19 do not rotate with respect to the core body 31. In each omnidirectional wheel 3, if a rotation speed difference occurs between the pair of drive discs 18, with respect to a force in the circumferential (tangential) direction due to rotation of the pair of drive discs 18, a force component in a direction orthogonal to this force acts on the driven roller 32 of the main wheel 19 from the left and right drive rollers 18B. Since an axis of the drive roller 18B is inclined with respect to the circumferential direction of the drive roller 18B, the force component is generated between the drive discs 18 due to the rotation speed difference. By this force component, the drive roller 18B is rotated with respect to the base 18A and the driven roller 32 is rotated with respect to the core body 31. Accordingly, the main wheel 19 generates a drive force in the left-right direction.

By rotation of the left and right omnidirectional wheels 3 forward at the same speed, the truck 1 moves forward. By rotation of the left and right omnidirectional wheels 3 rearward at the same speed, the truck 1 moves backward. By the occurrence of a speed difference in rotation of the left and right omnidirectional wheels 3 in the front-rear direction, the truck 1 turns to the right or to the left. By rotation of the driven roller 32 of each main wheel 19 of the left and right omnidirectional wheels 3, the truck 1 translates to the right or to the left.

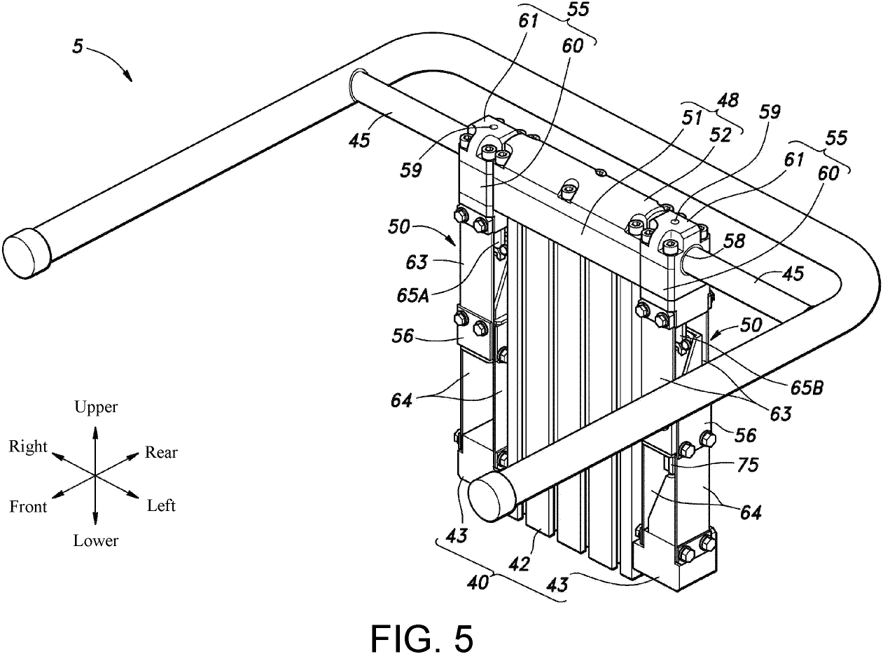

As shown in FIGS. 1 and 5, the operation device 5 is supported by the upper part of the rear part 2A of the body 2. The operation device 5 is provided with a base 40, an operation part 45, and a pair of left and right first connection parts 50.

The base 40 is provided with a first base part 42 connected to the body 2, and a pair of second base parts 43 connected to the first base part 42. The first base part 42 extends in an up-down direction. The front part of the first base part 42 is connected to the rear part 2A of the body 2. The pair of second base parts 43 are connected to side parts of the first base part 42.

The operation part 45 is disposed above the base 40 and extends in the left-right direction. A stopper 48 is provided between the operation part 45 and the first base part 42. The pair of left and right first connection parts 50 are provided between the operation part 45 and the pair of second base parts 43.

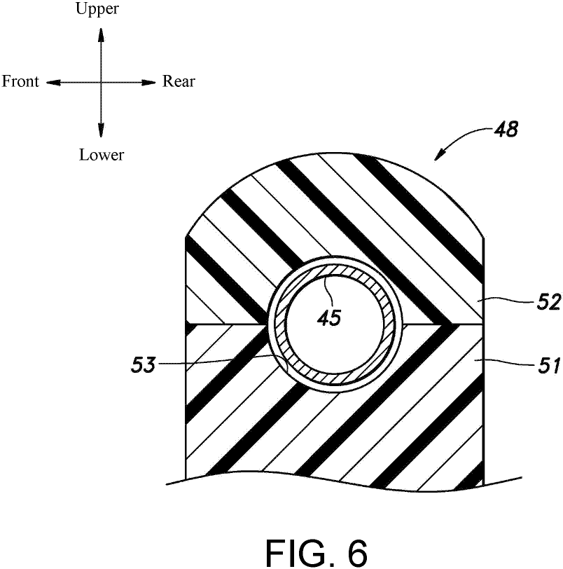

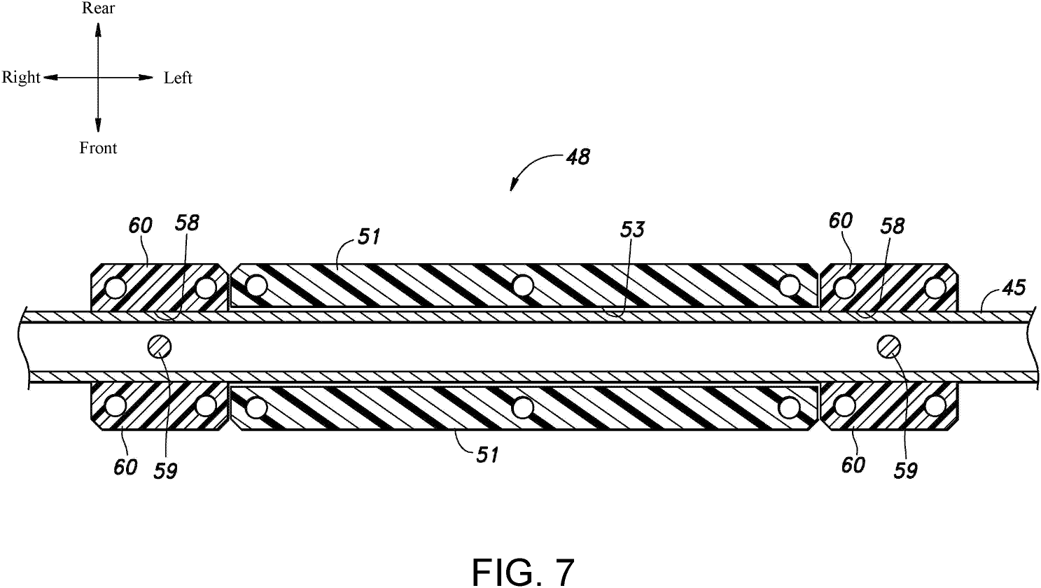

The stopper 48 is provided at the upper end of the first base part 42. The stopper 48 is provided with a lower stopper 51 and an upper stopper 52. The lower stopper 51 is connected to the first base part 42. The upper stopper 52 is connected to the upper part of the lower stopper 51. As shown in FIGS. 6 and 7, the upper surface of the lower stopper 51 and the lower surface of the upper stopper 52 define a first through hole 53 extending in the left-right direction. The first through hole 53 accommodates the central part of the operation part 45 in the left-right direction.

The operation part 45 is provided to be displaceable at least in the front-rear direction with respect to the stopper 48. When an operation force (load) is not applied to the operation part 45, the stopper 48 is disposed apart respect to the operation part 45 in the front-rear direction. When the user of the truck 1 (see FIG. 1) applies an operation force in the front-rear direction to the operation part 45, the operation part 45 abuts against the stopper 48 in the front-rear direction. The stopper 48 defines a movement range of the operation part 45 through the abutting against the operation part 45. As shown in FIG. 5, the pair of left and right first connection parts 50 are disposed on the left and right sides of the stopper 48.

The pair of left and right first connection parts 50 extend in the up-down direction, and are disposed apart from each other in the left-right direction. Each of the left and right first connection parts 50 is provided with a second end 55 connected to the operation part 45 and a first end 56 connected to the second base part 43. The first end 56 is connected to the second base part 45 by a second elastic member 64 to be described afterwards.

The second end 55 is located at the upper part of the first connection part 50. The second end 55 is provided with a body 60 and a cover 61 connected with the body 60. The cover 61 is connected to the upper part of the body 60. The upper surface of the body 60 and the lower surface of the cover 61 define a second through hole 58 extending in the left-right direction. As shown in FIGS. 5 and 7, the second through hole 58 accommodates the operation part 45.

The second end 55 and the operation part 45 are fastened by a pin 59. The pin 59 may be inserted downward from the top of the cover 61. Accordingly, the second end 55 is fixed with respect to the operation part 45 to be not displaceable in the front-rear direction and the left-right direction. In another embodiment, the second end 55 of the right first connection part 50 and the operation part 45 may also be displaceable in the left-right direction.

The first end 56 is located at the lower part of the first connection part 50 and above the second base part 43. The first end 56 is a member in a rectangular parallelepiped shape. The first end 56 and the second end 55 are connected by two first elastic members 63. The first end 56 and the second base part 43 are connected by two second elastic members 64.

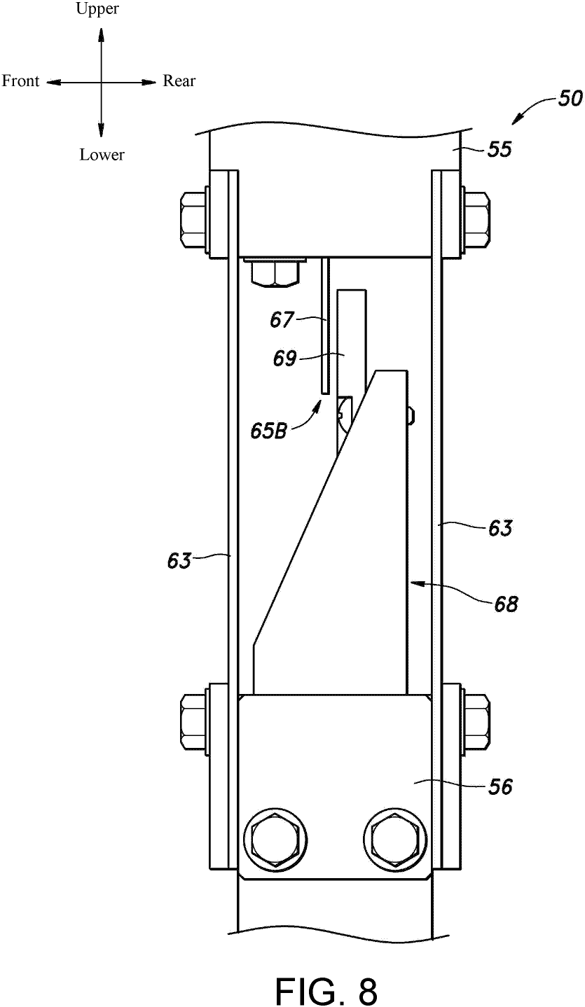

As shown in FIGS. 5 and 8, the two first elastic members 63 extend in the up-down direction. The two first elastic members 63 respectively have main surfaces facing forward and rearward, and are disposed apart from each other in the front-rear direction. The two first elastic members 63 include the first elastic member 63 on the front side and the first elastic member 63 on the rear side. The first elastic member 63 on the front side connects the front of the upper part of the first end 56 and the front of the lower part of the second end 55. The first elastic member 63 on the rear side connects the rear part of the upper part of the first end 56 and the rear part of the lower part of the second end 55. The first elastic member 63 may be formed by stainless steel. The first elastic member 63 connects the second end 55 to the first end 56 in a manner that is not displaceable in the left-right direction. The first elastic member 63 connects the second end 55 to the first end 56 in a manner that is displaceable in the front-rear direction.

The right first connection part 50 is provided with a first distance sensor 65A. The first distance sensor 65A is a sensor for measuring a relative distance (referred to as right first relative distance in the following) of the second end 55 with respect to the first end 56 in the front-rear direction.

The left first connection part 50 is provided with a first distance sensor 65B. The first distance sensor 65B is a sensor for measuring a relative distance (referred to as left first relative distance in the following) of the second end 55 with respect to the first end 56 in the front-rear direction. The first distance sensors 65A and 65B are each provided with a first piece 67 and a first eddy current sensor 69.

The first piece 67 is a conductive plate member. The first piece 67 functions as a measurement object of the first eddy current sensor 69. The first piece 67 is provided at a lower part of the second end 55. The first piece 67 extends downward toward the first end 56.

The first eddy current sensor 69 is provided at the upper part of the first end 56. The first eddy current sensor 69 may be supported by a first sensor support member 68 provided at the upper part of the first end 56. The first eddy current sensor 69 is disposed on the left of the first piece 67. The first eddy current sensor 69 is disposed at a position corresponding to the first piece 67 in the front-rear direction. The first eddy current sensor 69 is disposed at a position corresponding to the first piece 67 in the left-right direction.

The first eddy current sensor 69 is provided with a sensor coil and a resonant circuit. The sensor coil is connected to an AC power source and generates a magnetic field. When the first piece 67 is disposed in the magnetic field that is generated, an eddy current is induced. The magnitude of the eddy current changes in accordance with a distance (referred to as a first distance) between the first eddy current sensor 69 and the first piece 67. Specifically, as the first distance increases, the eddy current decreases. As the first distance decreases, the eddy current increases. Through the change of the magnitude of the eddy current, the impedance of the sensor coil changes. Accordingly, the output voltage of the resonant current also changes. Accordingly, the first distance can be detected from the change of the output voltage.

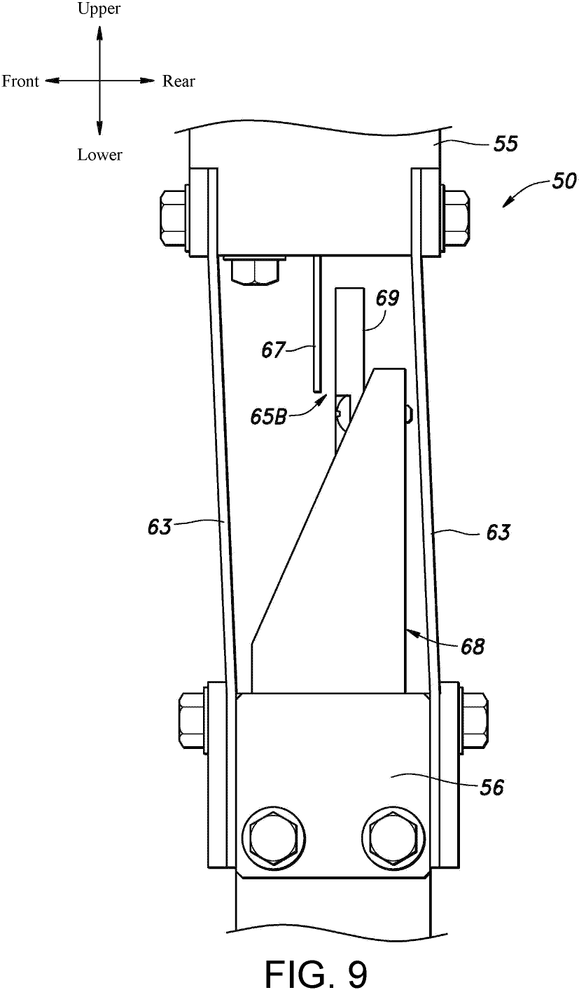

As shown in FIGS. 5 and 9, when the user applies a forward operation force to the operation part 45, a forward load is applied to the second end 55. The load to the second end 55 is transmitted to the first elastic member 63. Accordingly, the first elastic member 63 deforms, and the second end 55 is displaced forward with respect to the first end 56. Therefore, when the user applies a forward operation force to the operation part 45, the first distance increases. The first piece 67 is provided at the second end 55, and the first eddy current sensor 69 is provided at the first end 56. Accordingly, the first distance and the first relative distance are equal. Therefore, the first distance sensors 65A and 65B are able to detect the first relative distance.

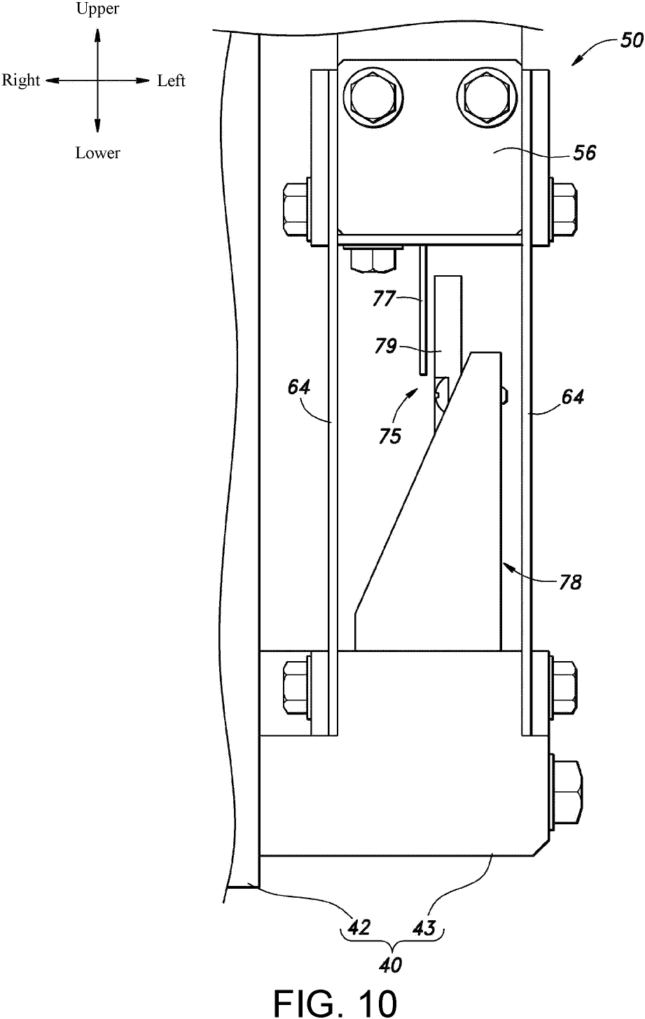

As shown in FIGS. 5 and 10, the two second elastic members 64 extend in the up-down direction. The two second elastic members 64 respectively have main surfaces facing leftward and rightward, and are disposed apart from each other in the left-right direction. The two second elastic members 64 include the right second elastic member 64 and the left second elastic member 64. The right second elastic member 64 connects the right side of the upper part of the second base part 43 and the right side of the lower part of the first end 56. The left second elastic member 64 connects the left side of the upper part of the second base part 43 and the left side of the lower part of the first end 56. The second elastic member 64 may be formed by stainless steel. The second elastic member 64 connects the first end 56 to the second base part 43 in a manner that is not displaceable in the front-rear direction. The second elastic member 64 connects the second base part 43 in a manner that is displaceable in the left-right direction.

The left first connection part 50 is provided with a second distance sensor 75. The second distance sensor 75 is a sensor for measuring a relative distance (referred to as second relative distance in the following) of the first end 56 with respect to the second base part 43 in the left-right direction. The second distance sensor 75 is provided with a second piece 77 and a second eddy current sensor 79.

The second piece 77 is a conductive plate member. The second piece 77 functions as a measurement object of the second eddy current sensor 79. The second piece 77 is provided at a lower part of the first end 56. The second piece 77 extends downward toward the second base part 43.

The second eddy current sensor 79 is provided at the upper part of the second base part 43. The second eddy current sensor 79 may be supported by a second sensor support member 78 provided at the upper part of the second base part 43. The second eddy current sensor 79 is disposed on the left of the second piece 77. The second eddy current sensor 79 is disposed at a position corresponding to the second piece 77 in the left-right direction. The second eddy current sensor 79 is disposed at a position corresponding to the second piece 77 in the front-rear direction. The configuration of the second eddy current sensor 79 is the same as the first eddy current sensor 69. Therefore, details in this regard will not be repeated.

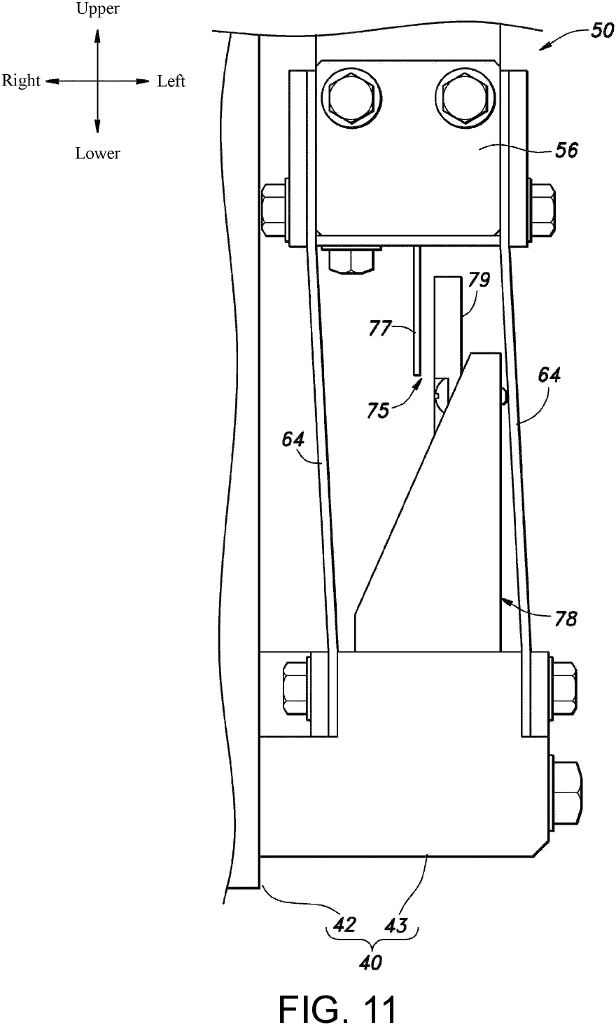

As shown in FIGS. 5 and 11, when the user applies a rightward operation force to the operation part 45, a rightward load is applied to the second end 55. The load to the second end 55 is transmitted to the second elastic member 64 via the first end 56. Accordingly, the second elastic member 64 deforms, and the first end 56 is displaced rightward with respect to the second base part 43. Therefore, when the user applies a rightward operation force to the operation part 45, the distance (referred to as second distance) between the second eddy current sensor 79 and the second piece 77 increases. The second piece 77 is provided at the first end 56, and the second eddy current sensor 79 is provided at the second base 43. Accordingly, the second distance and the second relative distance are equal. Therefore, the second distance sensor 75 is able to detect the second relative distance.

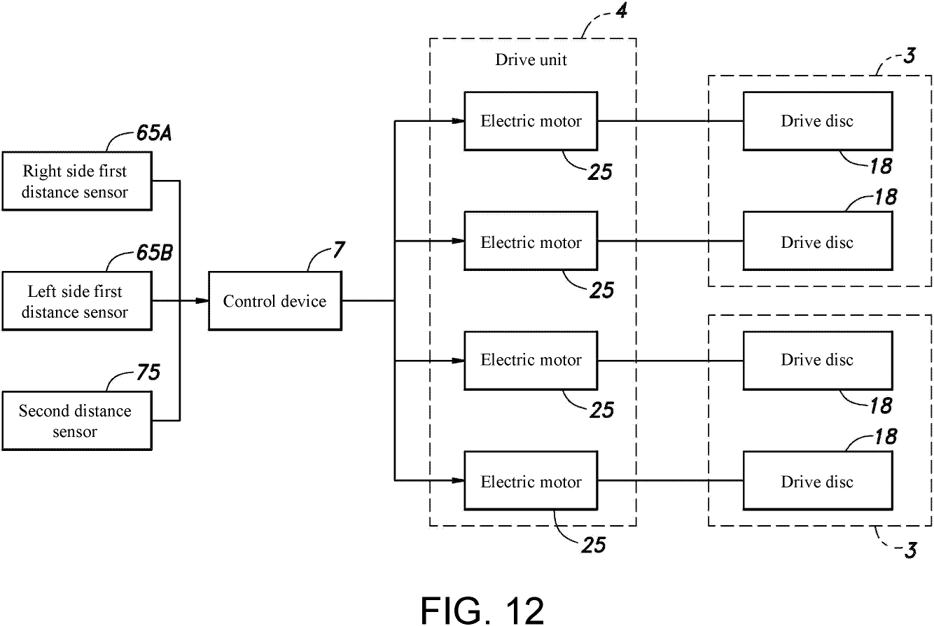

During the use of the truck 1 (see FIG. 1) by the user, the right side first distance sensor 65A detects the right first relative distance. The left side first distance sensor 65B detects the left first relative distance. The second distance sensor 75 detects the second relative distance. As shown in FIG. 12, the right side first distance sensor 65A, the left side first distance sensor 65B, and the second distance sensor 75 each output a signal indicating a detection result to the control device 7.

The control device 7 is an electronic control unit (ECU) including a processor such as CPU, a non-volatile memory (ROM) a volatile memory (RAM), and a hard disc drive (HDD), etc. The control device 7 controls the drive unit 4 by executing an arithmetic process in accordance with a program stored in the non-volatile memory through a processor. The control device 7 may be formed by one hardware component, and may also be formed as a unit including multiple hardware components. In addition, at least a portion of each functional part of the control device 7 may be realized by a hardware component, such as LSI, ASIC, FPGA, and may also be realized through software and hardware combination.

The control device 7 is connected with the right side first distance sensor 65A, the left side first distance sensor 65B, the second distance sensor 75, and the drive unit 4. The control device 7 controls the drive unit 4 based on the signals from the right side first distance sensor 65A, the left side first distance sensor 65B, and the second distance sensor 75. The control device 7 determines a control amount of each electric motor 25 based on the signals from the right side first distance sensor 65A, the left side first distance sensor 65B, and the second distance sensor 75. The control device 7 outputs the control signal indicating the control amount of each electric motor 25 to the drive unit 4. In the following, an example of a process in which the control device 7 controls the drive unit 4 is shown.

Initially, the control device 7 receives the signals from the right side first distance sensor 65A, the left side first distance sensor 65B, and the second distance sensor 75, and obtains the right first relative distance, the left first relative distance, and the second relative distance.

Then, the control device 7 acquires a right first reference value, a left first reference value, and a second reference value stored in advance in the storage device. The right first reference value is a distance between the first eddy current sensor 69 and the first piece 67 of the right side first distance sensor 65A when an operation force is not applied to the operation part 45. The left first reference value is a distance between the first eddy current sensor 69 and the first piece 67 of the left side first distance sensor 65B when an operation force is not applied to the operation part 45. The second reference value is a distance between the second eddy current sensor 79 and the second piece 77 of the second distance sensor 75 when an operation force is not applied to the operation part 45.

Then, the control device 7 calculates a displacement amount of the operation part 45 by comparing the first right relative distance, the left first relative distance, and the second relative distance with the corresponding reference values. Specifically, the control device 7 calculates a right first displacement amount from a difference between the right first relative distance and the right first reference value. The control device 7 calculates a left first displacement amount from a difference between the left first relative distance and the left first reference value. The control device 7 calculates a second displacement amount from a difference between the second relative distance and the second reference value.

Then, the control device 7 determines a moment msz based on a difference between the right first displacement amount and the left first displacement amount. The control device 7 determines a front-rear load fs1 based on a sum of the right first displacement amount and the left first displacement amount. The control device 7 determines a left-right load fs2 based on the second displacement amount.

For example, the control device 7 may also refer to a map in which the relationship between the difference between the right first displacement amount and the left first displacement amount and the moment msz is defined in advance to determine the moment msz. The front-rear load fs1 may also be determined by referring to a map in which the relationship between the sum of the right first displacement amount and the left first displacement amount and the front-rear load fs1 is defined in advance. The left-right load fs2 may also be determined by referring to a map in which the relationship between the second displacement amount and the left-right load fs2 is defined in advance. In addition, the control device 7 may also determine the moment msz, the front-rear load fs1, and the left-right load fs2 by using conventional formulae.

Then, the control device 7 sets a target front-rear speed vt1, a target left-right speed vt2, and a target angular speed ωt of the body 2. The control device 7 sets the target front-rear speed vt1 of the body 2 based on the front-rear load fs1. The control device 7 sets the target left-right speed vt2 of the body 2 based on the left-right load fs2. The control device 7 sets the target angular speed ωt of the body 2 based on the front-rear load fs1, the left-right load fs2, and the moment msz.

The control device 7, for example, may also refer to a map in which the relationship between the front-rear load fs1, the left-right load fs2, and the moment msz and the target front-rear speed vt1, the target left-right speed vt2, and the target angular speed ωt is defined in advance. The control device 7 may determine each of the target front-rear speed vt1, the target left-right speed vt2, and the target angular speed ωt based on reference results.

Then, the control device 7 sets a first rotation speed r1 of each electric motor 25 based on the target front-rear speed vt1. The control device 7 sets a second rotation speed r2 of each electric motor 25 based on the target left-right speed vt2. The control device 7 sets a third rotation speed r3 of each electric motor 25 based on the target angular speed ωt.

For example, the control device 7 may determine each of the rotation speeds r1, r2, and r3 by referring to a map in which the relationship between the target front-rear speed vt1, the target left-right speed vt2, and the target angular speed ωt and the rotation speed of each electric motor 25 is defined in advance.

Then, the control device 7 sets a target rotation speed rt (rt = r1+r2+r3) of each electric motor 25 by adding up the first rotation speed r1, the second rotation speed r2, and the third rotation speed r3 of each electric motor 25.

Then, the control device 7 sets a current value It supplied to each electric motor 25 based on the target rotation speed rt of each electric motor 25.

The control device 7, for example, sets the current value It by referring to a map in which the relationship between the target rotation speed rt and the current value It supplied to each electric motor 25 is defined in advance.

According to the above, each omnidirectional wheel 3 can be assist-driven by using the rotation speed of each electric motor 25 as the target rotation speed.

Then, effects of the operation device 5 according to the embodiments of the disclosure are described. When the user applies an operation force in the front-rear direction to the operation device 5, the left and right first elastic members 63 deform in the front-rear direction. Due to the deformation of the left and right first elastic members 63, the second ends 55 of the left and right first connection parts 50 are respectively displaced in the front-rear direction with respect to the corresponding first ends 56. The right side first distance sensor 65A detects the right first relative distance. The left side first distance sensor 65B detects the left first relative distance. By combining the right first relative distance and the left first relative distance, the first distance sensors 65A and 65B can detect an operation toward the front-rear direction and a turning operation performed by the user. Since the distance sensors can be acquired at a lower cost, an inexpensive operation device 5 can be provided.

In addition, the operation sensor 5 is at least provided with one second elastic member 64 and the second distance sensor 75. The second elastic member 64 connects each of the first ends 56 and the second base part 43 to be displaceable in the left-right direction. Accordingly, when the user performs an operation in the left-right direction, each of the first ends 56 is displaced with respect to the second base part 43 in the left-right direction. The second distance sensor 75 measures a relative distance of one of the first ends 56 with respect to the second base part 43 in the left-right direction. Accordingly, the operation force of the user in the left-right direction can be detected.

The second end 55 of the first connection part 50 disposed above the second distance sensor 75 (that is, the left first connection part 50) is fixed to the operation part 45 to be not displaceable in the left-right direction. Accordingly, the displacement of the operation part 45 in the left-right direction can be efficiently transmitted to the second elastic member 64.

The second end 55 of the first connection part 50 (i.e., the right first connection part 50) different from the first connection part 50 disposed above the second distance sensor 75 (that is, the left first connection part 50) is connected to the operation part 45 to be displaceable in the left-right direction. Accordingly, the measurement accuracy of the second distance sensor 75 can be facilitated.

The first distance sensors 65A and 65B are each provided with the first piece 67 that is conductive and the first eddy current sensor 69. The first piece 67 extends toward the corresponding first end 56 from the second end 55. The first eddy current sensor 69 is provided at the first end 56, and corresponds to the first piece 67 in the front-rear direction. In this aspect, the distance between the first eddy current sensor 69 and the first piece 67 is detected in accordance with the magnitude of the eddy current generated by the first eddy current sensor 69 and the first piece 67. Based on the detection result, the relative distance of the second end 55 with respect to the first end 56 in the front-rear direction can be detected. Since the eddy current sensor can be acquired at a lower cost, the first distance sensors 65A and 65B can be formed inexpensively. Moreover, by using the eddy current sensors, the sizes and the measurement distances of the first distance sensors 65A and 65B can be properly set.

Each second distance sensor 75 is provided with the second piece 77 that is conductive and the second eddy current sensor 79. The second piece 77 extends from the first end 56 toward the second base part 43. The second eddy current sensor 79 is provided at the second base part 43, and corresponds to the second piece 77 in the left-right direction. Since the eddy current sensor can be acquired at a lower cost, the second distance sensor 75 can be formed inexpensively. Moreover, by using the eddy current sensor, the environment resistance and the service time of the second distance sensor 75 can be facilitated.

The operation device 5 is further provided with the stopper 48 provided at the first base part 42. The stopper 48 defines a movement range of the operation part 45 through the abutting against the operation part 45. Accordingly, the operation force of the user can be suppressed from being applied to the first distance sensors 65A and 65B. Accordingly, the first piece 67 and the first eddy current sensor 69 can be prevented from contacting each and being damaged.

The stopper 48 is provided between the left and right second ends 55. The stopper 48 has the first through hole 53 extending in the left-right direction. The first through hole 53 accommodates the operation part 45. Accordingly, the left and right first contact parts 50 and the stopper 48 can be disposed in a compact manner.

Although the specific embodiments have been described above, the disclosure is not limited to the above embodiments and can be widely modified. For example, it may also be that at least one second elastic member 64 is connected to each second end 55 and the operation part 45 to be displaceable in the left-right direction. In such case, the second distance sensor 75 measures a relative distance of the operation part 45 with respect to one of the second ends 55 in the left-right direction. The second distance sensor 75 is disposed above the first distance sensor 65B. According to the configuration, the operation force of the user in the left-right direction can be detected.

In addition, although the first distance sensors 65A and 65B and the second distance sensor 75 are provided with the eddy current sensors 69 and 79, other sensors may also be provided. For example, it may also be that the first distance sensors 65A and 65B and the second distance sensor 75 are provided with non-contact sensors such as capacitive sensors, LED distance sensors, laser sensors, ultrasonic sensors, or contact sensors having contacts.

What is claimed is:

1. An operation device, comprising:
a base;
an operation part, extending in a left-right direction; and
a pair of left and right first connection parts, provided between the base and the operation part, and disposed apart from each other in the left-right direction,
wherein each of the first connection parts comprises:
a first end, connected to the base;
a second end, connected to the operation part;
a first elastic member, connecting the second end with respect to the first end to be displaceable in a front-rear direction; and
a first distance sensor, measuring a relative distance of the second end with respect to the first end in the front-rear direction.

2. The operation device as claimed in claim 1, comprising:
at least one second elastic member, connecting each of the first ends and the base to be displaceable in the left-right direction; and
a second distance sensor, measuring a relative distance of one of the first ends with respect to the base in the left-right direction.

3. The operation device as claimed in claim 2, wherein the second end of the first connection part disposed above the second distance sensor is fixed to the operation part to be not displaceable in the left-right direction.

4. The operation device as claimed in claim 2, wherein the second end of the first connection part different from the first connection part disposed above the second distance sensor is connected to the operation part to be displaceable in the left-right direction.

5. The operation device as claimed in claim 2, wherein each of the first distance sensors is provided with: a first piece, conductive and extending from the second end toward the corresponding first end; and a first eddy current sensor, provided at the first end and corresponding to the first piece in the front-rear direction.

6. The operation device as claimed in claim 2, wherein each of the second distance sensors is provided with: a second piece, conductive and extending from the first end toward the base; and a second eddy current sensor, provided at the base and corresponding to the second piece in the left-right direction.

7. The operation device as claimed in claim 1, further comprising a stopper, provided at the base and defining a movement range of the operation part through abutting against the operation part.

8. The operation device as claimed in claim 7, wherein the stopper is provided between the second ends at left and right, and
the stopper is provided with a through hole extending in the left-right direction and accommodating the operation part.

9. The operation device as claimed in claim 1, comprising:
at least one second elastic member, connecting each of the second ends and the operation part to be displaceable in the left-right direction; and
a second distance sensor, measuring a relative distance of the operation part with respect to one of the second ends in the left-right direction.

10. The operation device as claimed in claim 3, wherein each of the first distance sensors is provided with: a first piece, conductive and extending from the second end toward the corresponding first end; and a first eddy current sensor, provided at the first end and corresponding to the first piece in the front-rear direction.

11. The operation device as claimed in claim 4, wherein each of the first distance sensors is provided with: a first piece, conductive and extending from the second end toward the corresponding first end; and a first eddy current sensor, provided at the first end and corresponding to the first piece in the front-rear direction.

12. The operation device as claimed in claim 3, wherein each of the second distance sensors is provided with: a second piece, conductive and extending from the first end toward the base; and a second eddy current sensor, provided at the base and corresponding to the second piece in the left-right direction.

13. The operation device as claimed in claim 4, wherein each of the second distance sensors is provided with: a second piece, conductive and extending from the first end toward the base; and a second eddy current sensor, provided at the base and corresponding to the second piece in the left-right direction.

14. The operation device as claimed in claim 5, wherein each of the second distance sensors is provided with: a second piece, conductive and extending from the first end toward the base; and a second eddy current sensor, provided at the base and corresponding to the second piece in the left-right direction.

15. The operation device as claimed in claim 2, further comprising a stopper, provided at the base and defining a movement range of the operation part through abutting against the operation part.

16. The operation device as claimed in claim 15, wherein the stopper is provided between the second ends at left and right, and
the stopper is provided with a through hole extending in the left-right direction and accommodating the operation part.

17. The operation device as claimed in claim 3, further comprising a stopper, provided at the base and defining a movement range of the operation part through abutting against the operation part.

18. The operation device as claimed in claim 17, wherein the stopper is provided between the second ends at left and right, and
the stopper is provided with a through hole extending in the left-right direction and accommodating the operation part.

19. The operation device as claimed in claim 4, further comprising a stopper, provided at the base and defining a movement range of the operation part through abutting against the operation part.

20. The operation device as claimed in claim 19, wherein the stopper is provided between the second ends at left and right, and
the stopper is provided with a through hole extending in the left-right direction and accommodating the operation part.
